(19) United States
(12) Patent Application Publication
(10) Pub. No.: US 2013/0041441 A1
(43) Pub. Date: Feb. 14, 2013

(54) Hypothermia Management System

(71) Applicants: Darrel Saunders, Simpsonville, SC (US); Brent Bronson, Duncan, SC (US); Matt Westra, Simpsonville, SC (US)

(72) Inventors: Darrel Saunders, Simpsonville, SC (US); Brent Bronson, Duncan, SC (US); Matt Westra, Simpsonville, SC (US)

(73) Assignee: NORTH AMERICAN RESCUE, LLC, Greer, SC (US)

(21) Appl. No.: 13/209,384

(22) Filed: Aug. 13, 2011

(51) Int. Cl. A61F 7/08

(52) U.S. Cl. 607/114

(57) ABSTRACT

The objects of the invention are accomplished by providing a hypothermia management apparatus comprising: a receiving cavity defined by an outer shell arranged and sealable to encircle an injured person; an internal layer attached to the outer shell wherein the internal layer includes a bottom sheet disposed generally under the injured person when the injured person is placed in the receiving cavity and a top sheet disposed generally on top of the injured person when the injured person is placed in the receiving cavity; an inner liner attached to the bottom sheet of the internal layer forming an internal cavity; and, an absorption layer attached to the inner liner disposed generally under the injured person when the injured person is placed in the outer shell; and, a heater assembly disposed in the internal cavity.

### FIELD OF THE INVENTION

This invention is directed to a hypothermia management system for injured persons and more particularly, to a shell for receiving an injured person having a powered heater internally to maintain, but not exceed, a predetermined temperature inside the shell.

### BACKGROUND OF THE INVENTION

Based upon studies performed by the United State military, the number one cause for preventable combat death is one or more extremity hemorrhage. In fact, extremity hemorrhage accounts for approximately 60% of studied preventable combat deaths. When an injured person hemorrhages, hypothermia prevention and management is of critical importance. Hypothermia can lead to cardiac arrhythmias, decreased cardiac output, increased systemic vascular resistance, and most importantly, induced coagulopathy by inhibition of the clotting cascade. If hypothermia is not prevented in this casualty demographic, the potential exists that the injured person will not be able to form a clot due to the disruption of the clotting cascade and may potentially bleed to death from an otherwise survivable wounding.

The prior art discloses various means for treating and protecting against hypothermia including emergency blankets, insulated apparel, and heating devices, such as air activated heating pads. For example, U.S. Patent Publication No. 2005/0044602 discloses self-heating apparel having air activated heating elements sealed between layers of fabric and disposed throughout the apparel for providing a heating effect for the wearer. Another example is provided by U.S. Pat. No. 5,386,604, which discloses a patient rescue bag having top and bottom layers of insulated weatherproof material joined by releasable fasteners for adjusting the size of the bag to adapt to the size of the patient, while also keeping the patient warm and protected from the weather. Yet another example is provided by U.S. Pat. No. 4,998,296, which discloses a hypothermia protection suit having an integrated hood to be worn by a person in an emergency situation to provide extra warmth and protection from the weather. Another example is U.S. Pat. No. 7,766,950 which discloses hypothermia treatment sack having a plurality of self-activating heating pads carried in the top sheet for producing heat when exposed to air.

When dealing with the rigors of combat and the treatment and prevention of hypothermia in wounded patients, the prior art devices do not provide a sufficient solution to the unique problems encountered by medics on the battlefield. For example, the current practice by medics on the battlefield treating a patient with hypothermia is to first wrap the patient in a blanket containing air activated heating elements. These blankets are not heat reflective or weatherproof and are susceptible to rips and tears, and thus have been difficult to use in the field due to their fragility. Next, the medic will wrap the patient in a weatherproof heat reflective blanket, typically one made of lightweight aluminized polyester commonly referred to as a space blanket, around the first heating blanket to reflect the patient's body heat and heat from the blanket back against the patient. Finally, the medic will apply a heat reflective cap to the patient's head. The individual application of each of these items takes an unnecessarily long time. Worse, however, is the fact that by wrapping the patient in the heat reflective blanket, the air activated heating blanket does not get a sufficient supply of oxygen to sustain an acceptable level of heating capacity for extended time periods. Additionally, once the patient is wrapped in the blankets, the medic cannot easily check on the patient's wounds without unwrapping the patient, which exposes the patient and reduces the heat buildup around the patient intended to manage or prevent hypothermia.

Further, the heating devices that are air activated have little or no temperature control devices so that these heating devices provide heat in uncontrollable temperatures. Typically, these types of heating pads only provide for 8 to 12 hours of heating and generate temperatures between 122° F. to 131° F. (50° C. to 55° C.). However, it is medically recommended that heat applied to an injured person be approximately 104° F. (40° C.) to avoid further injury to the person.

Accordingly, it is an object of the present invention to provide a single use lightweight durable hypothermia treatment device having a controllable heat source.

It is another object of the present invention to provide a hypothermia treatment apparatus that can be powered by a variety of power sources eternal to the outer shell.

### SUMMARY OF THE INVENTION

The objects of the invention are accomplished by providing a hypothermia management apparatus comprising: a receiving cavity defined by an outer shell arranged and sealable to encircle an injured person; an internal layer attached to the outer shell wherein the internal layer includes a bottom sheet disposed generally under the injured person when the injured person is placed in the receiving cavity and a top sheet disposed generally on top of the injured person when the injured person is placed in the receiving cavity; an inner liner attached to the bottom sheet of the internal layer forming an internal cavity; and, an absorption layer attached to the inner liner disposed generally under the injured person when the injured person is placed in the outer shell; and, a heater assembly disposed in the internal cavity comprising: a heating substrate layer, a heating element adjacent the heating substrate layer, a power supply electrically connected to the heating element, a thermostat included in the electrical circuit for regulating the temperature produced by the heating element to a predetermined temperature, an electrical circuit defined by the heating element, the power supply and the thermostat, and a heater dissipating layer disposed adjacent the heating element for radiating heat into the outer shell at approximately 40° C.

### DESCRIPTION OF THE DRAWINGS

The invention will be more readily understood from a reading of the following specification and by reference to the accompanying drawings forming a part thereof, wherein an example of the invention is shown and wherein:

FIG. 1 is a perspectives view of the invention;

FIG. 2 is a top view of the invention;

FIG. 3 is a top view of a portion of the invention;

FIG. 4 is a cross-section view of the invention;

FIG. 5 is a cross-section view of a portion of the invention;

FIGS. 6A and 6B are schematics of aspects of the invention;

FIG. 7 is a perspective view of the invention;

FIG. 8 is a perspective view of the invention;

FIG. 9 is a top view of the invention; and,

FIG. 10 is a cross-section of the invention.

### DESCRIPTION OF A PREFERRED EMBODIMENT

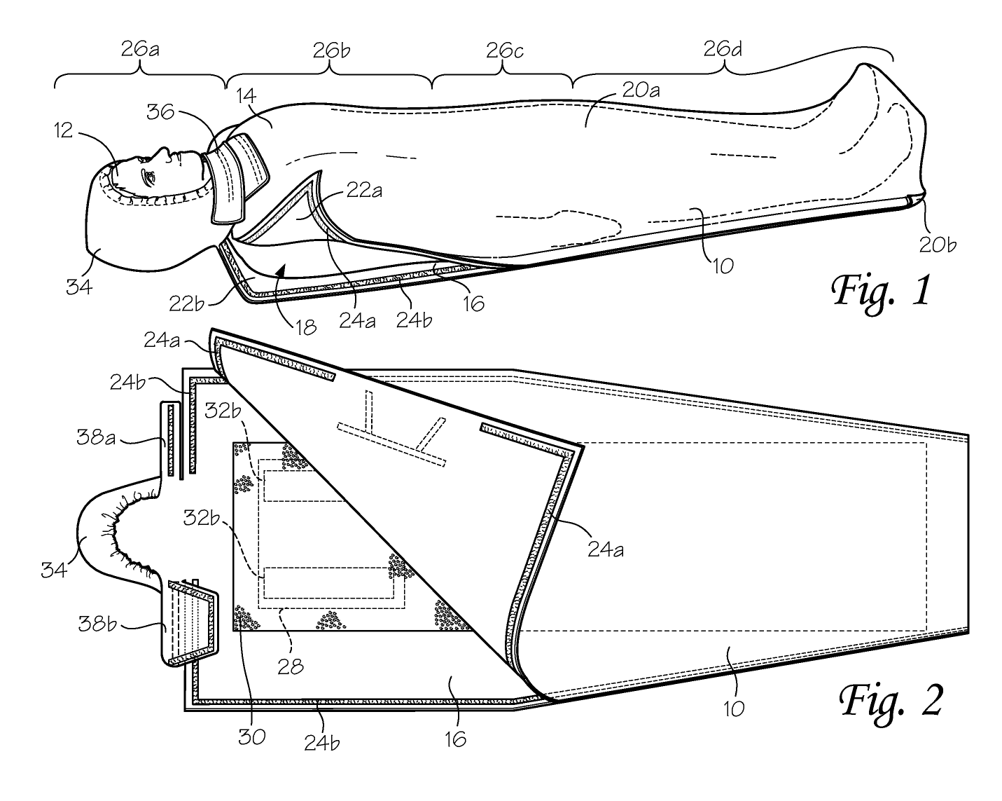

Referring to FIG. 1, an outer shell 10 is shown surrounding an injured person 12 for temporarily preventing and managing hypothermia. The outer shell is a durable cost efficient first aid device designed particularly for single use applications in the field by first responders to treat conditions associated with hypothermia by warming the patient and protecting the patient from inclement weather conditions until the patient can be transported to the appropriate treatment facility.

The outer shell includes a top sheet 14 and a bottom sheet 16 which can have corresponding rectangular shapes so that when the bottom sheet and top sheet are engaged, a receiving cavity 18 is formed. Top sheet 14 and bottom sheet 16 each have a weatherproof exterior side, designated generally as 20a and 20b, respectively, for repelling wind and water from entering patient receiving cavity 18. Further, top sheet 14 and bottom sheet 16 each have a heat reflective interior layer, designated generally as 22a and 22b, respectively, for preventing heat from escaping patient receiving cavity 18. In one embodiment, top sheet 14 and bottom sheet 16 comprise aluminized polyester which is capable of both repelling wind and water and reflecting heat. In this arrangement, only one sheet of materials is required to form each of top sheet 14 and bottom sheet 16, which reduces bulk and weight for storage and transport in the field. Further, aluminized polyester is extremely durable and well suited to the rigors of extreme field conditions, such on a battlefield.

In a further advantageous embodiment, weatherproof exterior sides 20a and 20b of top and bottom sheets 14 and 16, respectively, are a generally dark green color, such as olive drab, for camouflaging injured patients on a battlefield. In order to reduce manufacturing costs, heat reflective interior layer 22a and 22b may be the standard reflective finish common to aluminized polyester, which can be used to attract attention of rescuers if required.

When top sheet 14 and bottom sheet 16 are engaged to form patient receiving cavity 18, releasable connectors 24a and 24b are carried along a perimeter portion of top sheet 14 and bottom sheet 16 for releasably connecting the top and bottom sheets. This provides patient access from any location around the perimeter of the sheets when connected together. In a preferable embodiment, releasable connectors 24a and 24b comprise cooperating hook and loop fasteners, such as Velcro®. The receiving cavity can include a head area 26a, a chest area 26b, a torso area 26c, and a leg area 26d.

In one embodiment, a hood 34 is carried by bottom sheet 16 for covering a patient's head when placed in the receiving cavity. In a further embodiment, hood 34 includes a weatherproof exterior side for repelling wind and water from a patient's head. Further, hood 34 can include a heat reflective internal layer for preventing heat from escaping through the hood. As with top sheet 14 and bottom sheet 16, hood 34 may be constructed from aluminized polyester to provide lightweight durable wind and water resistance while also providing heat reflective capability.

In one embodiment, a neck closure, designated generally as 36, is carried by hood 34 and adapted for surrounding a patient's neck. Preferably, the neck closure includes a weatherproof exterior side for repelling wind and water from the patient's neck, and a heat reflective interior liner for reducing heat loss from the patient's neck. Neck closure 36 can include a first flap 38a including a first portion of hook and loop fastener, and a second flap 38b including a second portion of cooperating hook and loop fastener for engaging hook and loop fastener of first flap, wherein first flap and second flap overlap each other around a patient's neck.

Referring to FIG. 2, releasable connectors 24a and 24b are arranged into strips extending continuously along the perimeter of the top sheet and bottom sheet. A heating assembly 28 can be included in the chest area 26b and disposed between the bottom sheet and an absorption pad 30. The heating assembly can include heating elements 32a and 32b. In one embodiment, the heating assembly is generally in a square arrangement and completely contained within the chest area. The absorption pad can be of a non-woven material such as a felt, in one embodiment 4 oz felt, or a layers absorption pad with outer absorption material enclosing a chemical absorption material such as a polymer, chemically crosslinked cellulose fiber, sodium polyacrylate and the like. The absorption layer, in one embodiment, extends from the chest area to the leg area within the receiving cavity. It is advantageous when placing an injured person in the outer shell that any fluids excreted by the injured person are absorbed and wicked away from the injured person.

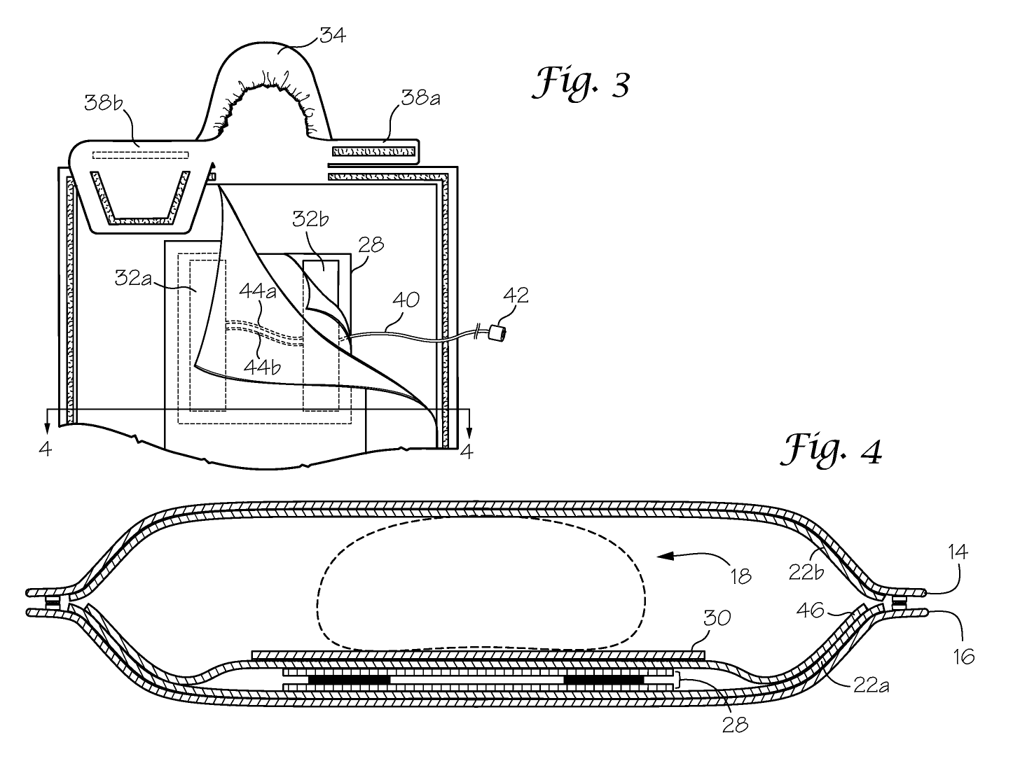

Referring to FIG. 3, heating elements 32b and heating element 32a are electrically connected by a negative lead 44a and a positive lead 44b. Since the heating elements are conductive, an electrical circuit is formed. Power supply cable 40 is connected to the heating elements and is attached to a power supply terminal 42.

Referring to FIG. 4, a cross-section of the invention is shown. Top sheet 14 of the outer shell includes a top internal layer 22b. The internal liner can be an internal side of the outer shell or displayed internally in said top sheet. Bottom sheet 16 can include interior layer 22a. Heater assembly 28 is adjacent to the interior liner 22a. An inner liner 46 can be attached to the outer shell enclosing the heater assembly between the inner liner and the bottom sheet of the outer shell. The inner liner can be heat reflective or a lightweight non-woven fabric such as felt. Absorption pad 28 is attached to the inner liner.

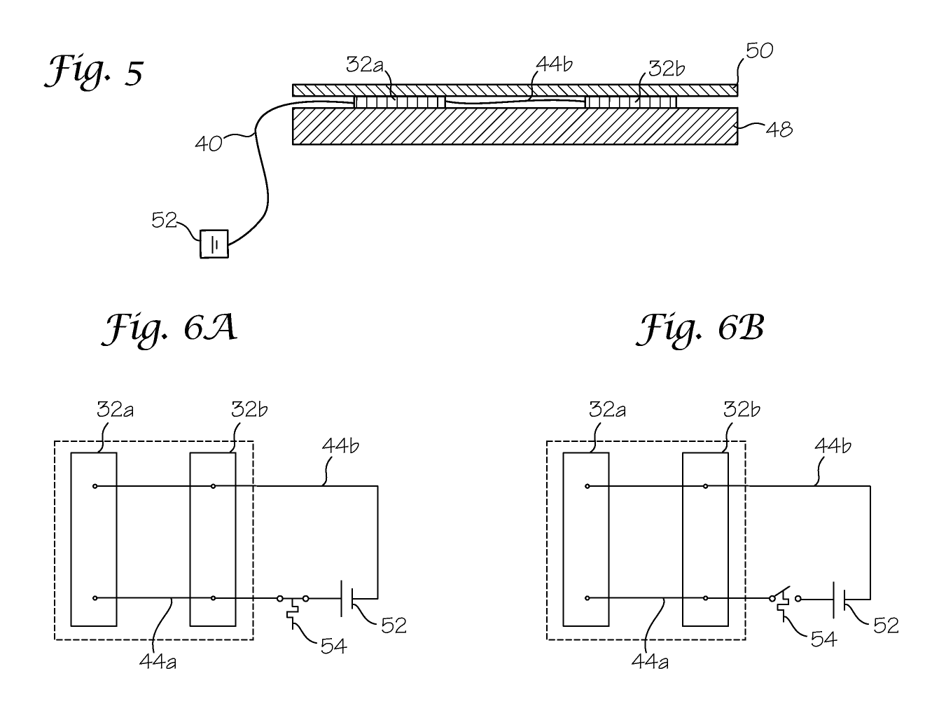

Referring to FIG. 5, the heating assembly is shown in more detail. Heating substrate layer 48 carries heating elements 32a and 32b. Heating dissipation layer 50 is adjacent to the heating elements with the positive and negative leads disposed between the heater substrate layer and the heating dissipation layer. Power supply cable 40 extends from between the heater substrate layer and the heating dissipation layer so that it can connect to power supply 52.

In one embodiment, heater substrate layer and heating dissipation layer are non-woven materials such as felt. Further, the heater substrate material can be of 12 oz density and the heating dissipation layer can be a 4 oz density. The heating elements can generate temperatures which are dissipated by the dissipating layer. For example, if the heating elements produce temperatures in the range of 55° C. to 65° C., the heat will be dissipated after passing through the dissipation layer to a range including 35° C. and 45° C. Therefore, the optimal temperature for the receiving cavity of 40° C. can be maintained by the dissipating layer.

Referring to FIGS. 6A and 6B, the heating assembly is shown in more detail. An electric circuit is shown including power supply 52, a thermostat 54, power connectors (positive lead 44a and negative lead 44b) and heating elements 32a and 32b. The thermostat can be configured so that it is in a closed position as shown in FIG. 6A when the ambient temperature surrounding the heating elements is less than approximately 60° C. Therefore, the heat delivered to the receiving cavity, after passing through the dissipation layer, would be approximately 40° C., the preferred temperature. When the ambient temperature surrounding the heating elements is less than approximately 60° C., the thermostat can move to an open position as shown in FIG. 6B thereby opening the electrical circuit until the ambient temperature drops below 60° C.

In one particular embodiment, the heating element is a thin-film consisting of a relatively thin polyamide, or polyester film impregnated with a resistive carbon layer onto which conductive busses (leads) are printed. The leads can be comprised of low resistance, highly conductive materials such as silver, copper or gold. The film of the heater element itself is less than 0.5 mm thick with strength, flexibility and reliability. In one particular embodiment the heater is laminated with a 0.1 mm layer of Teflon™ to allow for forming heater into a wave pattern. Critically, this heater technology draws far less power than competitive alternatives allowing for extended battery life and lightweight, highly portable designs. In one exemplary embodiment, the heater material of the heater elements may be directly applied to the housing members using a spray and/or deposition technique. In one alternate embodiment, it is possible to use a single layer carbon-based resistive film that is directly applied to the outer surface of the inner housing.

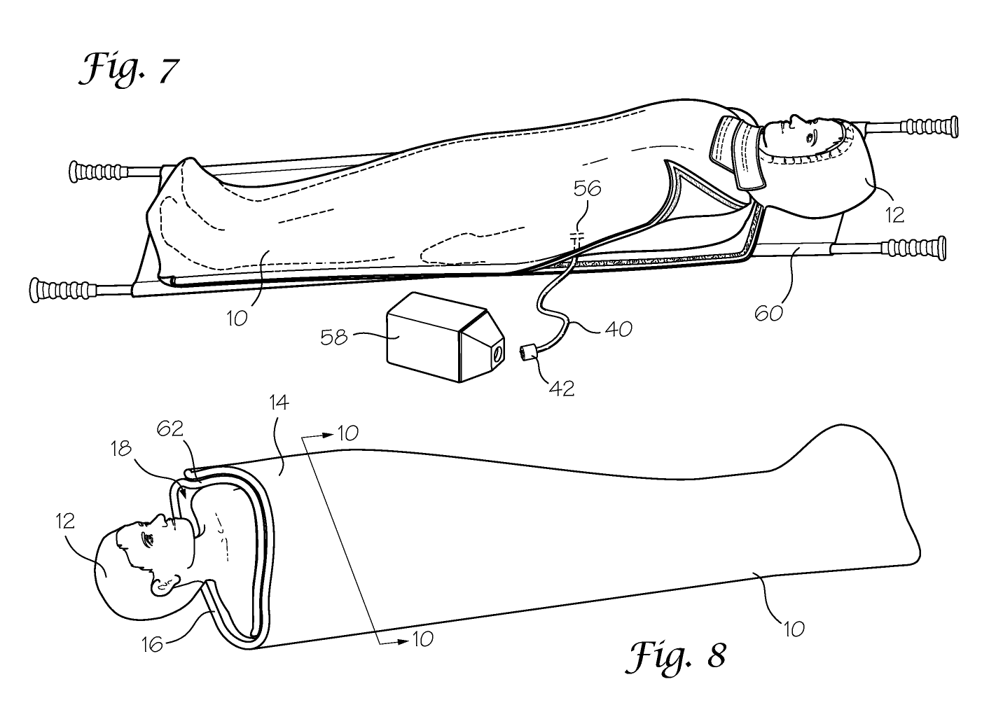

Referring to FIG. 7, the outer shell with an injured person in the receiving cavity, can be placed on a litter 60 or the like. The power cable can exit the outer shell through cable opening 56 so that the power supply terminal can be connected to an external power such as battery 58. In one embodiment, the power source is a battery having the designation BA-5590. The power supply terminal can be a BA-5590 connector. Having interchangeable power supplies allows the treatment of hypothermia to last as long as a power source is available, unlike air activated heating elements. In one embodiment, heating element 32a is arranged in series with heating elements 32b. In one embodiment, heating element 32a is arranged in parallel with heating element 32b so that if heating element 32a malfunctions, an electrical circuit still exists defined by heating element 32b, the thermostat, and the power supply. Because the heating elements are conductive, they complete the electrical circuit.

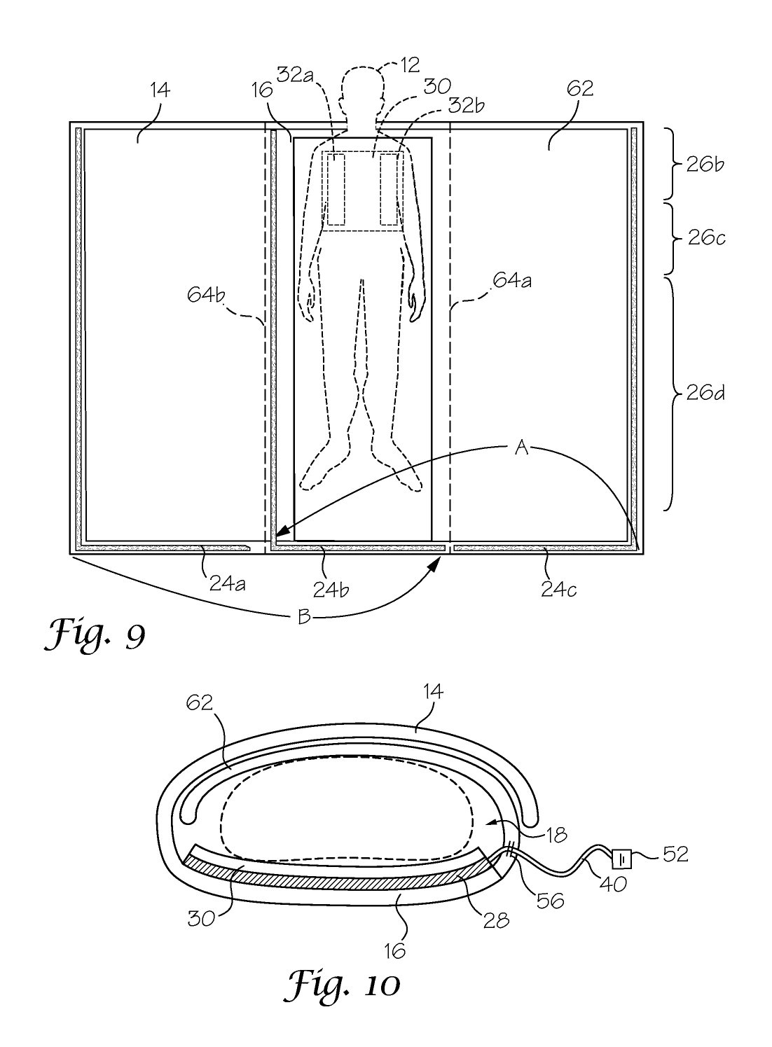

Referring to FIG. 10, another embodiment of the invention is shown. The injured person is shown in receiving cavity 18 which is formed by bottom sheet 16, a top sheet (for first top sheet) 14 and second top sheet 62. Referring to FIG. 9, the injured person is placed on bottom sheet 16 with heating assembly 28 disposed generally under the back or torso of the injured person. Second top sheet 62 is then folded over at fold 64a and placed on top of the injured person. Second top sheet 62 can be releasably secured to bottom sheet 16 by releasable connectors 24c and 24b. Top sheet 14 can then be folded over the top of the injured person along fold 64b so that top sheet 14 is laying on top of second top sheet 62. Top sheet 14 can be releasably secured to outer side of second top sheet 62 by releasable connectors 24a and releasable connectors (not shown) attached to the outer side of second top sheet. Referring to FIG. 10, receiving cavity 18 is shown defined by bottom sheet 16, top sheet 14 and second top sheet 62. Heating assembly 28 is shown disposed between bottom sheet 16 and absorption layer 30.

The invention can be packaged within a vacuum sealed pliable container capable of maintaining a high gas barrier while sealed.

While a preferred embodiment of the invention has been described using specific terms, such description is for illustrative purposes only, and it is to be understood that changes and variations may be made without departing from the spirit or scope of the following claims.

What is claimed is:

1. A hypothermia management apparatus comprising:
a receiving cavity defined by an outer shell arranged and sealable to encircle an injured person;
an internal layer attached to said outer shell wherein said internal layer includes a bottom sheet disposed generally under the injured person when the injured person is placed in said receiving cavity and a top sheet disposed generally on top of the injured person when the injured person is placed in the receiving cavity;
an inner liner attached to said bottom sheet of said internal layer forming an internal cavity; and,
an absorption layer attached to said inner liner disposed generally under the injured person when said injured person is placed in said outer shell;
a heater assembly disposed in said internal cavity comprising:
a heating substrate layer,
a heating element adjacent said heating substrate layer,
a power supply electrically connected to said heating element,
a thermostat included in said electrical circuit for regulating the temperature produced by said heating element to a predetermined temperature,
an electrical circuit defined by said heating element, said power supply and said thermostat, and
a heater dissipating layer disposed adjacent said heating element for radiating heat into said outer shell at approximately 40° C.

2. The apparatus of claim 1 wherein said internal layer is a heat reflective layer for preventing heat from escaping said receiving cavity.

3. The apparatus of claim 1 including:
a chest area, torso area and leg area included in said receiving cavity; and
said heating assembly is contained within said chest area.

4. The apparatus of claim 1 wherein said thermostat remains in a closed position completing said electrical circuit as long as the temperature generated by said heating elements remains less than approximately 60° C., else said thermostat is in an open position.

5. The apparatus of claim 1 wherein said heating substrate layer has a density of approximately 12 oz and said heater dissipating layer has a density of approximately 4 oz.

6. The apparatus of claim 1 wherein said dissipation layer prevents heat from entering the receiving cavity to be higher than approximately 40° C.

7. The apparatus of claim 1 including:
a power supply cable having a first end connected to said heating element and a second end connected to a power supply terminal; and,
a cable opening in said outer shell allowing said power supply cable to be attached to a power supply external to said outer shell.

8. The apparatus of claim 7 wherein said power supply terminal is a BA-5590 connector.

9. The apparatus of claim 1 wherein said absorption layer is a 4 oz felt.

10. The apparatus of claim 1 including a second heating element included in said electrical circuit.

11. The apparatus of claim 10 wherein said second heating element is arranged in parallel in said electrical circuit with said heating element.

12. A hypothermia management apparatus comprising:
an outer shell defining a receiving cavity for receiving an injured person, said outer shell including a bottom sheet disposed generally under the injured person and a top sheet disposed generally on top of the injured person when the injured person is in the receiving cavity;
an inner liner attached to said bottom sheet forming an internal cavity between said inner liner and said outer shell;
an absorption layer attached to said inner liner disposed generally under the injured person when said injured person is placed in said receiving cavity; and,
a heating assembly disposed in said internal cavity having a heating substrate layer, a heating element adjacent said heating substrate layer, a power supply connected to said heating element, a thermostat connected to said power supply for regulating the temperature produced by said heating element and a heater dissipating layer disposed adjacent said heating element.

13. The apparatus of claim 12 including an internal heat reflective layer attached to the interior of said outer shell for preventing heat from escaping said receiving cavity;

14. The apparatus of claim 12 including:
a chest area, torso area and leg area included in said receiving cavity; and
said heating assembly is contained within said chest area.

15. The apparatus of claim 12 wherein:
said thermostat remains in a closed position completing said electrical circuit as long as the temperature generated by said heating elements remains less than approximately 60° C., else said thermostat is in an open position; and,
said dissipation layer prevents heat from entering the receiving cavity to be higher than approximately 40° C.

16. The apparatus of claim 12 including a second heating element included in an electrical circuit defined by said heating element, said second heating element, said thermostat and said power supply.

17. The apparatus of claim 16 wherein said second heating element is arranged in parallel in said electrical circuit with said heating element

18. A hypothermia management apparatus comprising:
an outer shell arranged to wrap around an injured person, said outer shell having a bottom sheet disposed generally under the injured portion when said outer layer is wrapped around the injured person and a first top sheet disposed generally on top of the injured person;
an absorption layer attached to said bottom sheet; and,
a heating assembly disposed between said bottom sheet and said absorption layer having a heating substrate layer, a heating element adjacent to said heating substrate layer, a power supply connector for connecting said heating element to a power supply, a thermostat for regulating the temperature produced by said heating element and a heater dissipating layer disposed adjacent said heating element.

19. The apparatus of claim 18 including a second top sheet included in said outer shell disposed generally on top of said first top sheet when said second top sheet is wrapped around the injured person.

20. The apparatus of claim 18 including:
a receiving cavity defined by said outer shell when said outer shell is wrapped around the injured person; and,
said outer shell is a heat reflective material for preventing heat from escaping said receiving cavity.

21. The apparatus of claim 18 including:
a receiving cavity defined by said outer shell when said outer shell is wrapped around the injured person;
a chest area, torso area and leg area included in said receiving cavity; and
said heating assembly is contained within said chest area.

22. The apparatus of claim 18 wherein:
said thermostat remains in a closed position completing an electrical circuit as long as the temperature generated by said heating elements remains less than approximately 60° C., else said thermostat is in an open position; and,
said dissipation layer prevents heat from entering the receiving cavity to be higher than approximately 40° C.

23. The apparatus of claim 18 including a second heating element included in said heating assembly.

24. The apparatus of claim 23 wherein said second heating element is arranged in parallel with said heating element in an electrical circuit defined by said heating element, said second heating element, a power supply and said thermostat.

25. The apparatus of claim 18 including:
a power supply cable included in said power supply connector having a first end connected to said heating element and a second end connected to a power supply terminal; and,
a cable opening in said outer shell allowing said power supply cable to be attached to a power supply external to said outer shell.

26. The apparatus of claim 25 including a power supply connected to said power supply terminal.

27. The apparatus of claim 26 wherein said power supply is a BA-5590.
